(19) United States
(12) Patent Application Publication
(10) Pub. No.: US 2020/0196942 A1
(43) Pub. Date: Jun. 25, 2020

(54) METHOD AND SYSTEM FOR MONITORING A SUBJECT IN A SLEEP OR RESTING STATE

(71) Applicant: SAN DIEGO STATE UNIVERSITY RESEARCH FOUNDATION, San Diego, CA (US)

(72) Inventors: Yusuf OZTURK, San Diego, CA (US); Ahmet Enis CETIN, Chicago, IL (US)

(21) Appl. No.: 16/613,388

(22) PCT Filed: May 15, 2018
(86) PCT No.: PCT/US2018/032841

§ 371 Date: Nov. 13, 2019

Related U.S. Application Data
(60) Provisional application No. 62/507,174, filed on May 16, 2017.
(60) Provisional application No. 62/592,394, filed on Nov. 29, 2017.

(51) Int. Cl. A61B 5/00; A61B 5/024; A61B 5/113; A61B 7/00; A61B 5/0205

(52) U.S. Cl. CPC A61B 5/4815; A61B 5/0077; A61B 5/02416; A61B 5/4812; A61B 5/1135; A61B 5/7264; A61B 2562/0219; A61B 5/0205; A61B 5/7257; A61B 5/7465; A61B 5/6887; A61B 2562/0204; A61B 7/003

(57) ABSTRACT

Methods and systems for monitoring a subject in a sleep or resting state are provided herein. The methods can include obtaining signals from at least one of one or more microphones, one or more pyroelectric infrared (PIR) sensors and one or more accelerometer sensors. The obtained signals can be automatically transmitted to a processor. Thereafter, using the processor, one or more patterns in the obtained signals can be detected to determine one or more physiological and/or biological parameters including at least one of heart rate, breathing rate, wheezing, sleep quality and/or sleep architecture. Outputs of the determined parameters may be generated upon crossing preset thresholds.

### RELATED APPLICATIONS

This application claims the filing date of previously filed provisional applications 62/507,174 entitled “Heart Rate Detection and Monitoring using Pyroelectric Infrared (PIR) Sensor,” filed May 16, 2017, and 62/592,394, entitled “Microphone Embedded Blanket for Heart-rate and Wheezing Detection,” filed Nov. 29, 2017; the contents of both of which are incorporated herein by reference.

### FIELD

Embodiments of the present application relate to methods and systems of monitoring a subject during a sleep or rest state; and more specifically to methods and systems including non-intrusive sensors configured to determine one or more physiological and/or biological parameters of a subject.

### BACKGROUND

Accurate measurement and monitoring of physiological parameters play an important role in a broad range of applications in the field of healthcare, psycho-physiological examinations and sports training, for example. When it comes to measuring fitness and monitoring potential health issues, there are few numbers more helpful or convenient than your resting heart rate (RHR). While a normal RHR or pulse differs from one individual to another, if one keeps track of it over time, it can elicit some important data regarding one's health and fitness.

RHR is measured by beats per minute (BPM). For the super fit, RHR tends to be lower because a healthy heart can pump more blood with each beat with greater efficiency, thus requiring fewer beats per minute to pump blood throughout the body. Conversely, an elevated heart rate can be a sign of health issues.

RHR is a particularly useful measurement because the numbers are not influenced by outside factors in the same way as if you were to take your heart rate after exercise or a long day of work. When one takes his or her RHR as soon as he or she wakes up, one is more likely to be calm and unencumbered by stress the way one might be later in the day. This is also typically the time when one's heart will be pumping the lowest volume of blood because he or she is at rest.

During sleep, response to external stimuli is reduced. Sleep is a state that is characterized by changes in brain wave activity, breathing, heart rate, body temperature, and other physiological functions. Sleep is usually divided into Rapid Eye Movement (REM) and Non-Rapid Eye Movement (Non-REM) stages.

During REM sleep, all muscles cease to move except the eyes. During this stage eyes moves rapidly while all muscles are paralyzed. REM stage accounts for about 25% of each night's sleep. REM sleep is the period during which psychological repair of the cells takes place. Dreams that take place during REM sleep are remembered.

Non-REM sleep has three stages, namely Non-REM1, Non-REM2 and Non-REM3 stages. Non-REM1 stage is the first stage where sleep onset occurs and the sleep is the lightest. This stage accounts for 5% of the sleep duration. Non-REM2 stage is the deeper sleep stage, which accounts to 45% of the sleep duration. Finally, Non-REM3 stage, also known as Slow Wave Sleep (SWS), is the deepest stage of the sleep. This stage amounts to 25% of the total sleep duration. The rest of the sleep period is classified as the REM sleep during which time all movement stops.

Sleep architecture refers to the basic structural organization of normal sleep and reports the overall progression of nightly sleep. In a normal sleep cycle, one moves from the lighter stages of sleep (Non-REM1) to deeper stages (Non-REM2 and Non-REM3) before moving into the REM sleep and a return to lighter sleep before a new cycle begins. The sleep cycle repeats itself roughly every 90 minutes. Over the night the composition of the sleep stages will differ. During the initial cycle, one will spend very short periods of time in REM sleep and comparatively more time in Non-REM sleep stages. As the night progresses sleep cycles gradually changes extending periods of REM sleep and reducing periods of deeper Non-REM sleep.

At each stage of the sleep, the body is engaged in repairing certain aspect of the body. During REM sleep, the body replenishes the energy to the brain and the body. During Non-REM sleep blood supply to the muscles increases, muscles are relaxed and tissue repair occurs. During Non-REM sleep growth hormones which are essential for growth and development are released. Tracking sleep and understanding the how you sleep is important since getting enough sleep is essential for developing and maintaining a healthy mind and body.

Tracking sleep allows for determining if a subject is sleeping enough and whether one's sleep disturbances are a result of a sleep disorder. Sleep apnea is one of the most commonly pronounced sleep disorder which disrupt the sleep continuously resulting in feeling of tiredness through the day and may lead to more serious conditions such as Alzheimer's disease. Among other causes of sleep disorders stress, obesity, insomnia, sleepwalking, difficulty falling asleep can be listed as examples. Understanding the sleep architecture and measuring the sleep quality is the first step in diagnosis and cure of sleep disorders or underlying causes.

Conventionally, sleep stage identification and sleep assessment are performed using polysomnography (PSG) which combines several biophysiological measurements to determine sleep stages. It is usually performed at night, when most people sleep, in a sleep laboratory. The PSG monitors many body functions including brain, eye movements, muscle activity or skeletal muscle activation, and heart rhythm during sleep. To diagnose sleep apnea breathing functions respiratory airflow and respiratory effort indicators are measured along with peripheral pulse oximetry for monitoring blood oxygen levels. PSG is an expensive process requiring a night in a sleep laboratory in a controlled environment hooked up to various measurement systems (electrical and flow measurements). It has been viewed conventionally as essential for diagnosis of certain sleep disorders thus the cost and the discomfort have been justified.

Sleep monitoring today is often performed by using wearable devices which are mostly worn on the wrist. Some wristbands monitor sleep, and there are even specialized devices that go on your head or bedside table that can also keep track of how long and how well you sleep each night. Although wearable devices are in abundance today, for sleep tracking they are not for everyone. Many people who often wear fitness trackers during the day, do not want to go to sleep in them due to discomfort.

Non-contact techniques, on the other hand, are non-intrusive and more adequate for long-term monitoring. Despite some shortcomings, the nonintrusive nature makes them an attractive option for daily monitoring. Several techniques based on laser doppler, microwave doppler radar, ultra-wideband radar, frequency modulated continuous wave radar and thermal imaging have been investigated. The advantage of these approaches is that they do not require users to wear any sensors on their bodies; however, as they rely on outwardly expressed states, they are susceptible to interference by external factors.

### SUMMARY

Embodiments described herein are directed to monitoring a subject in a sleep or resting state through the use of a set of non-contact and non-intrusive sensors. According to one embodiment, a method of monitoring a subject in a sleep or resting state is disclosed, which includes obtaining signals from at least one of one or more microphones, one or more pyroelectric infrared (PIR) sensors and one or more accelerometer sensors. The obtained signals can be automatically transmitting to a processor, which can be used for detecting one or more patterns in the obtained signals to determine one or more physiological and/or biological parameters including at least one of heart rate, breathing rate, wheezing, sleep quality and/or sleep architecture.

According to an embodiment at least a subset of the at least one of the one or more microphones, one or more PIR sensors and one or more accelerometers sensors can be incorporated as part of a blanket used by the subject.

Another embodiment is directed to system for monitoring a subject in a sleep or resting state. The system can include at least one of one or more microphones, one or more PIR sensors and one or more accelerometer sensors obtaining signals. A wireless transmitter automatically transmits the obtained signals to a processor, which is configured to detect one or more patterns in the obtained signals to determine one or more physiological and/or biological parameters including at least one of heart rate, breathing rate, wheezing, sleep quality and/or sleep architecture.

Yet another embodiment is directed to system for monitoring a subject in a sleep or resting state, including a blanket used by the subject and including at least one of one or more microphones and one or more accelerometer sensors. The system can include one or more PIR sensors, and a wireless transmitter communicatively coupled to the blanket and the one or more PIR sensors automatically transmitting obtained signals from the at least one of the one or more microphones, the one or more accelerometer sensors and the one or more PIR sensors to a processor. According to an embodiment, the processor is configured to detect one or more patterns in the obtained signals to determine one or more physiological and/or biological parameters including at least one of heart rate, breathing rate, wheezing, sleep quality and/or sleep architecture.

Yet another embodiment is directed to a blanket for monitoring a subject in a sleep or resting state. The blanket can include at least one of one or more microphones, and one or more accelerometer sensors obtaining signals when the blanket is used by the subject. The blanket can further include a wireless transmitter automatically transmitting the obtained signals to a processor, which is configured to detect one or more patterns in the obtained signals to determine one or more physiological and/or biological parameters including at least one of heart rate, breathing rate, wheezing, sleep quality and/or sleep architecture.

Yet another embodiment is directed to an apparatus for monitoring a subject in a sleep or resting state. The apparatus can include one or more PIR sensors, and a wireless transmitter communicatively coupled to the one or more PIR sensors automatically transmitting obtained signals from the one or more PIR sensors to a processor. According to certain embodiments, the processor is configured to detect one or more patterns in the obtained signals to determine one or more physiological and/or biological parameters including at least one of heart rate, breathing rate, wheezing, sleep quality and/or sleep architecture.

The embodiments disclosed herein are further capable of generating at least one of an audio output, a visual output, an alert message, a report or any combinations thereof, upon determining that values of the determined physiological and biological parameters are beyond corresponding thresholds.

Within the scope of the present disclosure, one or more transitory or non-transitory computer readable media are disclosed, storing instructions thereon for, when executed by a processor, performing any of the functions described herein.

Various other features and advantages will become obvious to one of ordinary skill in the art in light of the following detailed description and drawings.

### BRIEF DESCRIPTION OF THE DRAWINGS

The accompanying drawing figures incorporated in and forming a part of this specification illustrate several aspects of the disclosure, and together with the description serve to explain the principles of the disclosure.

FIG. 1 shows exemplary data obtained from a single PIR sensor, according to an exemplary embodiment.

FIG. 2 shows an exemplary system with a subject sleeping near PIR sensors, microphones and accelerometers, according to an exemplary embodiment.

FIGS. 3(a)-3(c) show PIR data obtained from chest movement of a subject, according to an exemplary embodiment.

FIG. 4 shows an experimental setup of how data is collected via PIR sensor from a subject in a rest state, according to an exemplary embodiment.

FIGS. 5(a) and 5(b) show an exemplary PIR sensor signal and its low-pass filtered version, according to an exemplary embodiment.

FIGS. 6(a) and 6(b) show exemplary low-pass filtered data and the corresponding 2^{nd} order derivative signal, according to an exemplary embodiment.

FIGS. 7(a) and 7(b) show a 2^{nd} order derivative signal and zero-crossings, according to an exemplary embodiment.

FIG. 8 shows a scatter plot of PIR sensor measurements of resting heart rate compared to PPG sensor measurements, according to an exemplary embodiment.

FIG. 9 shows a cumulative distribution function of the difference between estimated PIR sensor values and PPG sensor values, according to an exemplary embodiment.

FIG. 10 is an exemplary flowchart illustrating a method of monitoring a subject in a sleep or resting state, according to an exemplary embodiment.

### DESCRIPTION OF EMBODIMENTS

In the following description, numerous specific details are set forth. However, it is understood that embodiments of the invention may be practiced without these specific details. In other instances, well-known circuits, structures and techniques have not been shown in detail in order not to obscure the understanding of this description. Those of ordinary skill in the art, with the included descriptions, will be able to implement appropriate functionality without undue experimentation.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether or not explicitly described.

In the following description and claims, the terms “coupled” and “connected,” along with their derivatives, may be used. It should be understood that these terms are not intended as synonyms for each other. “Coupled” is used to indicate that two or more elements, which may or may not be in direct physical or electrical contact with each other, co-operate or interact with each other. “Connected” is used to indicate the establishment of communication between two or more elements that are coupled with each other.

The embodiments set forth below represent information to enable those skilled in the art to practice the embodiments and illustrate the best mode of practicing the embodiments. Upon reading the following description in light of the accompanying figures, those skilled in the art will understand the concepts of the disclosure and will recognize applications of these concepts not particularly addressed herein. It should be understood that these concepts and applications fall within the scope of the disclosure.

Embodiments of the present disclosure described herein relate to health monitoring, and more particularly to sound and/or motion analysis-based methods and systems for non-invasive and non-contact monitoring of a subject in a resting or sleep state. Monitoring such physiological parameters during sleep, for example, may reveal important information about the health status of the subject.

Pyroelectric Infrared (PIR) sensors are widely used for sensing motion of subjects. Infrared radiation exists in the electromagnetic spectrum at a wavelength that is longer than visible light. Objects that generate heat also generate infrared radiation that remains invisible to the human eye but can be measured by electronic sensors. PIR sensors can detect the levels of infrared radiations and thus are commercially used for automation of electrical appliances and home surveillance systems, for example, by detecting radiation emitted by humans or animals.

The basic functionality of differential PIR sensor is to measure the difference in infrared radiation density of two pyro-electric elements within the sensor. Normal variations in the temperature caused by the air are nullified by the two elements connected to a differential amplifier. If the elements measure the same amount of infrared radiation, a differential amplifier produces an output of zero. If different levels of heat are detected by the sensors, the differential amplifier will report a nonzero value indicating infrared radiation source motion.

Most of the commercially available PIR motion sensor circuits produce digital output. Nonetheless, analog signal output can also be obtained from PIR sensors. As described herein, one can exploit the analog signal obtained from the PIR sensor to detect chest motion as a result of breathing and eventually estimate sleep quality using a combination of algorithms that monitor different levels and types of motion generated by a subject's whole body, parts of the body and chest motion during a sleep episode, for example.

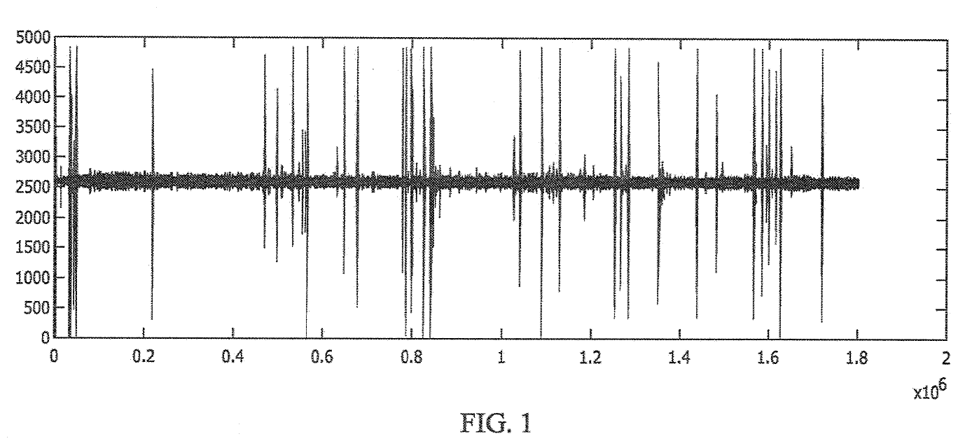

FIG. 1 shows exemplary data obtained from a single PIR sensor placed next to (or near) a bed on a bedside table or other apparatus positioned in a manner that the sensor can obtain movement information of a subject, for example. The large amplitude swings of the data of FIG. 1 correspond to large body movements while the suppressed amplitude parts correspond to the stages where sleep is taking place, in the present embodiment.

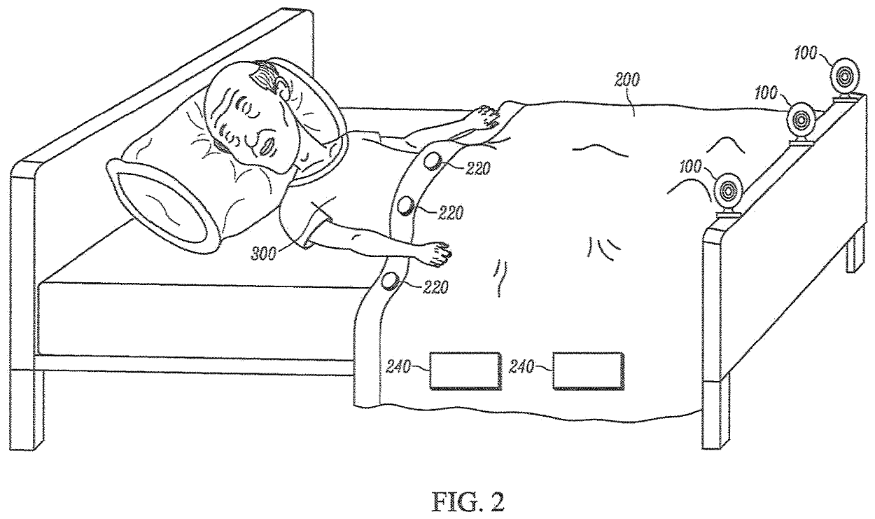

One of ordinary skill in the art would realize that various positions of the PIR sensor or sensors can be implemented to obtain the appropriate data. For example, one or more sensors can be placed on a bedside table embedded in décor or a lamp. FIG. 2 shows an exemplary embodiment with a subject 300 lying in a bed under a blanket 200. The PIR sensors 100 in this example are coupled to the footboard of the bed and, thus, can non-intrusively obtain radiation readouts to determine movement of subject 300 during a sleep state, for example. The exemplary embodiment of FIG. 2 shows three PIR sensors 100; however, any number could be implemented within the scope of the present disclosure.

Using one or an array of sensors, two or more persons sleeping in the same bed can be monitored, for example. The wireless sensors 100 acquire the data and send the data to a sleep monitor application running on a smart phone to be incorporated to other biophysical markers in a smart health application. The first step in processing to analyze sleep data and estimate the quality of sleep is the segmentation stage where the data is segmented into motion and sleep areas. Once the segmentation is performed a low pass filter is applied on sleep segments to extract the respiration rate by monitoring the periodic chest movements, as discussed in further detail below. Although not depicted, it should be noted that the PIR sensor(s) 100 can incorporate various antennae, transceivers and processor(s) to implement various types of wireless communication technology, such as Bluetooth and/or WiFi, and/or cellular wireless communication.

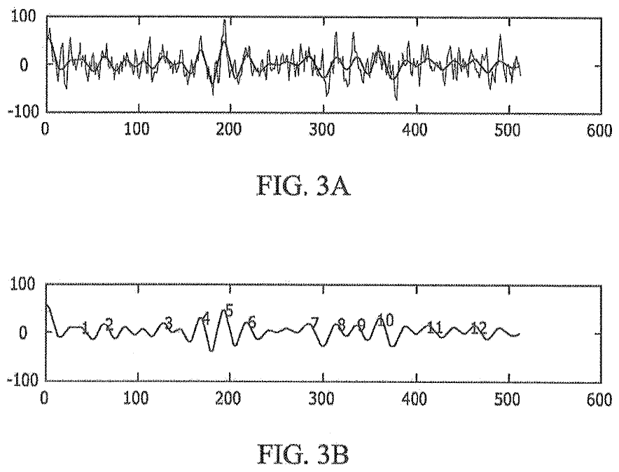

FIGS. 3(a)-3(c) present 512-point chest movement data from a sleep segment representing 51.2 seconds of sleep of a subject. FIG. 3(a) shows a segment of data determined to be during sleep by the segmentation algorithm. The data is low pass filtered using a Discrete Cosine Transform (DCT) and after elimination of nearly 90% of the higher order coefficients, in this example, the filtered signal is transformed back into time domain. This low pass filtered signal is shown in FIG. 3(b), which is also overlaid on top of the original signal in FIG. 3(a). The slow-moving, near periodic wave is due to the chest motion as a result of respiration.

There are high frequency oscillations riding the low frequency respiration related chest movements. The low frequency oscillations can be extracted and the high frequency oscillations are plotted in FIG. 3(c). These high frequency oscillations are highly correlated with the heart rate.

To assess sleep quality the sleep data can be segmented into several segments of non-movement (not necessarily sleep) and movement (not necessarily wake) segments. The primary reason for this segmentation is the ability to monitor smaller vibrations triggered by chest movements alone during moments of no movement. The sleep data can be analyzed to (1) determine total sleep time, (2) sleep latency, (3) sleep efficiency, (4) wake after sleep onset, (5) awakening index. These parameters summarized can form the bases if a metric named sleep quality index.

For example, methods and systems herein can determine if a person (a) goes to sleep within 30 minutes after going to bed, (b) wakes up at various times during the night, (c) leaves the room to go the bathroom at night, (d) cannot breathe comfortably, (e) coughs or snores loudly, (f) stays in bed for a particular amount of time, and/or (g) gets a particular amount of sleep at night. Exemplary items (a)-(g) are the quantitative parameters of the Pittsburgh Sleep Quality Index (PSQI). A person can fill the questionnaire of the PSQI more accurately using the parameters determined by the methods and systems described herein.

For example, the PIR sensor(s) 100 can measure parameter (a) because the subject 300 stops moving after he or she falls asleep. The PIR sensor(s) 100 can determine parameters (b) and (c) if a subject 300 wakes up at night and leaves the room because the sensor signal output will be much larger and longer than movements during sleep. Microphone(s) 220 embedded to the blanket, for example, can determine parameters (d)-(e), e.g., by determining if a subject 300 coughs or not, snores or not and/or has difficulty breathing or not. Also, by analyzing the data of accelerometer(s) 240 and the PIR sensor(s) 100 methods and the systems described herein can determine the amount of time the subject 300 stayed in bed and percentage of the time the subject 300 is asleep. For example, before the subject 300 goes to bed and after the subject 300 leaves the bed both the accelerometer(s) 240 and PIR sensor(s) 100 do not record any motion activity. Thus, it can be determined how long the subject 300 is in bed.

The methods and systems described herein can also measure the various sleep stages identified by amplitude and periodicity, by outputting sensor data and patterns as described herein. Large movements can also be seen as peaks spanning several seconds. Summaries of an awake-state determination and each sleep stage as determined are given below:

Awake/movement—Clear and strong peaks indicate physical movement. Consecutive peaks indicate being awake. Meanwhile, single, lonely peaks indicate movement during sleep. A cluster of consecutive peaks with time in between indicates leaving and returning to bed. FFT analysis shows a strong peak at 0.2-0.3 during sleep stages and random distribution during awake and silent stages.

REM Stage—This stage is identified with highly varying breathing patterns. Regions of high variability indicate REM sleep. It is similar to a silent segment in this regard. It has a low breathing volume and amplitude. Amplitude is significantly lower than deep sleep. Early NREM stages (light sleep) exhibit slightly greater amplitude and lower variability.

NREM 1&2 (Light Sleep)—The variability rate is much lower than REM but closer to deep sleep. Amplitude is lower than deep sleep but slightly higher than REM. Light sleep also acts as a transition stage. For example, it is uncommon to see direct transition between REM and deep.

NREM 3&4 (Deep Sleep)—This is a simple determination, with very low variability and high signal amplitude. This stage tends to follow immediately after light sleep.

### Data Acquisition

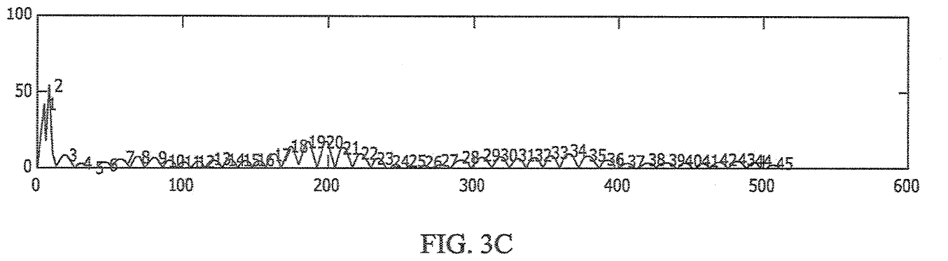

In the case of a subject 300 at rest, but not asleep, the data is collected using one or more PIR sensors 100, as shown in FIG. 4. The analog output of the PIR sensor 100 can be sampled with a sampling rate of 10 Hz for higher, according to an exemplary embodiment, using a microcontroller and the resulting signal is fed to a personal computer, or any other processing device capable of outputting information to a user. The resulting signal can also be digitized and sent to a cloud processor or a mobile device for processing and presentation to the user via a graphical easy to use interface. The use of a laptop, desktop, wireless handheld device or any other system could be implemented to obtain the resulting signal, within the scope of the present disclosure.

The second-order derivative of the discrete-time PIR sensor 100 can be computed by a processor at the person computer, etc. to extract the heart beat signal. Of course, the processing of the signal can be performed any processor, which could be a remote cloud processor or a local processor. A zero-crossing or a peak detection algorithm, for example, can be then applied to the resultant output to estimate RHR. The PIR sensor 100 detects the chest motion, caused by the inhale-exhale process and the resting heart rate, to provide an analog signal, as described above. The chest motion is a resultant of two physiological processes: respiration and heartbeat vibrations. The respiratory activity is, however, much larger in magnitude in comparison to the heartbeat vibrations, as described with respect to FIGS. 3(a)-3(c). On the other hand, the acceleration of the breathing activity is much lower than the acceleration of the heartbeat vibrations.

Computing the second-order derivative of the signal with respect to time provides us a result related with the acceleration of the chest of subject 300. As a result, the second order derivative signal must be mainly due to the heartbeat activity. The impulse response of the most widely used first-derivative filter is:

h[n]=[1 0 −1] [1]

The corresponding transfer function is:

H(z)=z+z⁻¹ [2]

By convolving this filter with itself, we get the second-order derivative filter,

h₂[n]=[1 0 −2 0 1] [3]

However, the filter h₂[n] potentially cannot be used to estimate the resting heart rate, because the recorded data from the PIR sensor 100 may be noisy. Therefore, the data can be smoothed with a simple Lagrange low pass filter with an impulse response of [1 2 1] before applying the second-order derivative filter. Since the sampling frequency of the PIR signal is 10 Hz, the full band is 5 Hz. The LPF is an approximate half-band filter, i.e., it attenuates the high frequency components above 2.5 Hz. Therefore, it does not affect the RHR of a person. The main effect of the LPF is to remove small ripples in the signal which may be due to A/D conversion.

The equivalent impulse response becomes:

g₂[n]=[1 2 −1 −4 −1 2 1] [4]

Since the Lagrange low-pass filter [1 2 1] is also a triangular window, it is possible to scale the window size before applying the second-order derivative filter h₂[n]. Convolving the data with a wider triangular window [1 4 6 4 1] may provide even better noise cancellation. Consequently, the effective impulse response of the present filter becomes:

g′₂[n]=h₂[n]*[1 4 6 4 1] [5]

or

g′₂[n]=[1 4 4 −4 −10 −4 4 4 1] [6]

Normalized version of the corresponding input/output relationship is given by

y[n]=(10x[n]+4(x[n−1]+x[n+1])−4(x[n−2]+x[n+2])−4(x[n−3]+x[n+3])−(x[n−4]+x[n+4]))/36 [7]

The filter given in Eq. [7] can produce a better result than the filter given by Eq. [4]. The computational load of the heart-rate estimation algorithm is low because the FIR filter given in Eq. [7] has integer coefficients. Therefore, the overall system can be implemented using a low cost digital signal processor or any general micro-controller.

### Experimental Setup

In an experimental setup, a total of 30 subjects 300 were tested between 20 to 55 years old. The lab environment included furniture which includes tables, desks, chairs and computers. During the experiment PIR sensor is placed on a table about 1 meter away from subject 300. FIG. 4 shows an exemplary experimental setup similar to those conducted in the lab environment. The subject 300 is instructed to breath in and out as he/she does regularly, during which the chest movements are captured. The results were compared to the industry standard photoplethysmogram (PPG) sensor and the estimated values match with the photoplethysmogram (PPG) sensor.

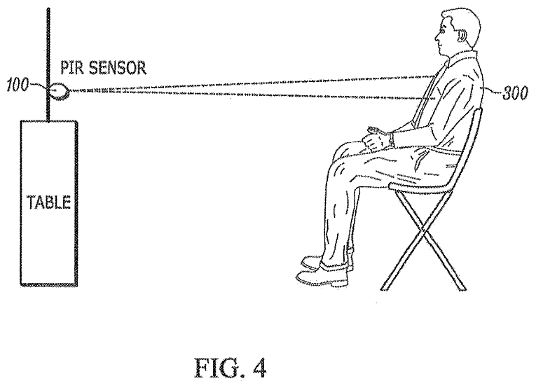

FIGS. 5(a)-5(b) shows the PIR sensor 100 signal output due to chest motion of one of the subjects 300 who is about 1 meter away from the sensor 100 (FIG. 5(a)) and the output after low pass filtering (FIG. 5(b)). As it can be seen from FIGS. 5(a)-5(b), the PIR sensor signal is almost periodic due to breathing patterns. Therefore, it is possible to estimate the breathing rate from the PIR sensor signal.

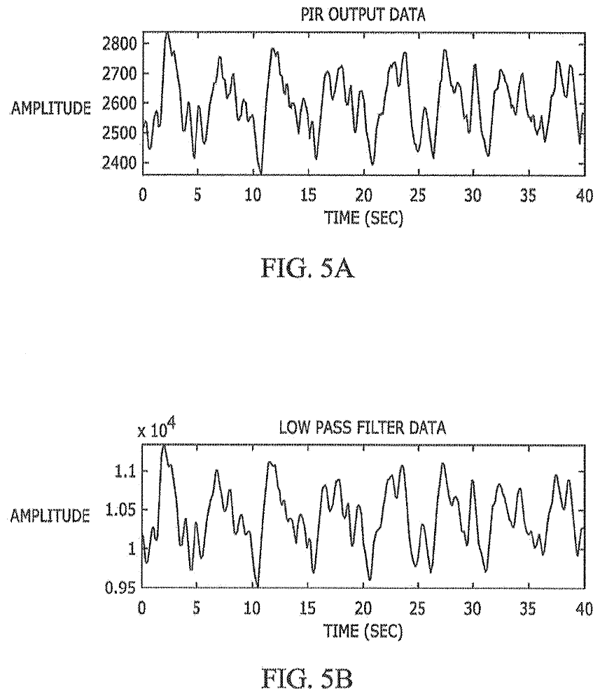

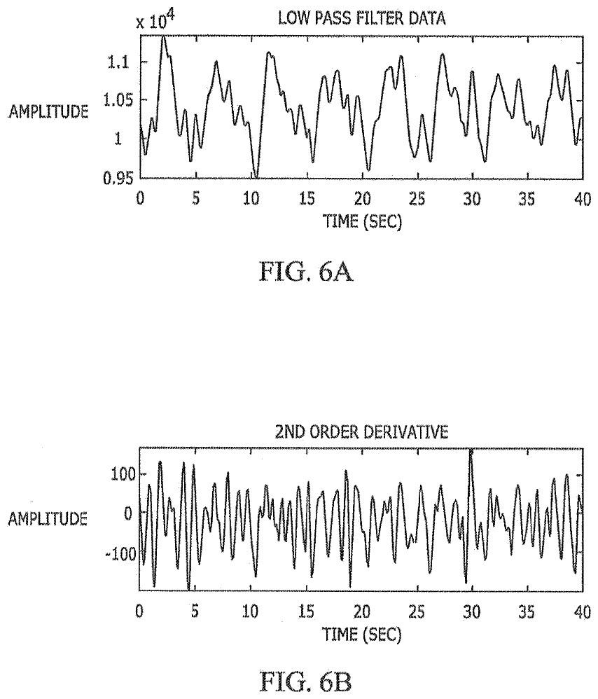

FIGS. 6(a)-6(b) show the output of the 2nd order derivative filter and FIGS. 7(a) and 7(b) shows zero crossings in the output signal. By counting the zero crossings of the 2nd order derivative signal per minute one can estimate the RHR. Since this subject 300 has 20 zero crossings in 10 seconds his RHR is estimated at 60 beats per minute.

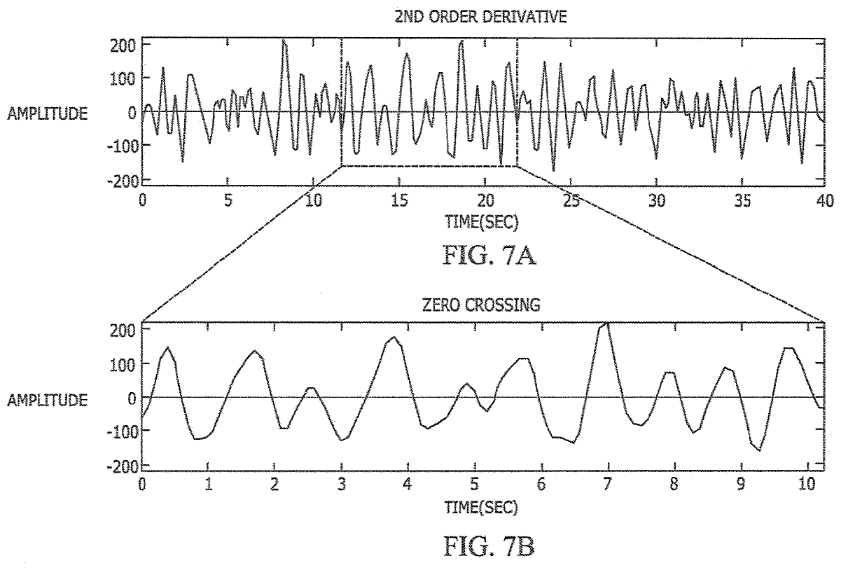

A total of 60 experiments were conducted, collecting over 10,000 heart beats. During the experiment each subject is simultaneously monitored with a PIR sensor 100, as described in the present disclosure, and a PPG device. The estimated RHR values and the industry standard RHR values obtained using the PPG sensor are shown in FIG. 8, which was obtained from 30 subjects. From each subject two PIR records were collected. The horizontal axis represents the experiment number and vertical axis represents the heart rate in BPM. FIG. 8 shows the scatter plot between heart rate values of the PPG device and the PIR sensor 100, where x-axis represents heart rate values of the PPG device and y-axis represents heart rate values of the PIR sensor. As shown in FIG. 8, there is a direct correlation between the two sensors.

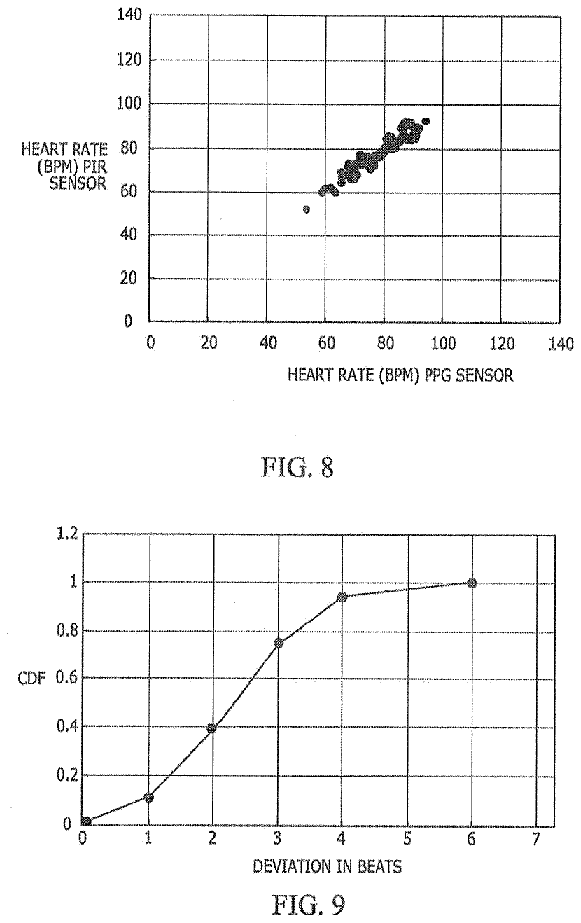

FIG. 9 shows a cumulative distribution function (CDF) of the differences between estimated PIR sensor 100 values and the industry standard PPG sensor. The CDF shows that 95% of values have deviation less than 4 beats per minute. Results further show that the mean values of the 60 RHR (30 subjects, two measurements per subject) measurements using the PPG and the PIR sensors 100 are 75.5 BPM (beats per minute) and 74.7 BPM (beats per minute), respectively. To assess the validity of the experiments, a chi-square test is employed:

$${X}^{2} = {\sum }_{i = 1}^{k} {( {O}_{i} - {E}_{i} )}^{2} \text{/} {E}_{i} \qquad [ 8 ]$$

where Oᵢ is heart rate values from the PIR sensor 100, Eᵢ is heart rate values from the PPG sensor, and k=60. The estimated heart rate values from the PIR sensor 100 are reliable with a significance level of α=0.05.

For even further analysis of additional biological and physiological markers, additional or alternative sensors can be implemented, within blanket 200 of FIG. 2, for example. For example, built-in microphone(s) 220 can capture sounds the subject 300 makes, including breathing sounds, heart sounds, whizzing, wheezing, etc. Additional motion detectors, such as accelerometers 240, may be incorporated into such a system as well. Of course, the number of sensors and their placements can be configured in many different ways, as should be understood to one of ordinary skill in the art. In some embodiments, some sensors, including one or more microphones 220, may be positioned separate from blanket 200. A processor for analyzing the data obtained from microphones 220 and/or accelerometers 240 can be similarly incorporated into blanket 200, or may be remote at a user work station (e.g., laptop) that obtains signals from sensors via wireless communication, for example, as described above, in order to output data to a user.

### Respiration Rate Detection

Respiration is a quasi-periodic behavior corresponding to the number of breaths taken per minute. The normal respiration rate for an adult at rest is 12 to 20 breaths per minute. Therefore, the respiration sound is also periodic. The present disclosure may apply the average magnitude detection function (AMDF) to the sound data captured by microphone(s) 220, for example. Assuming that the sound data is sampled and a discrete-time signal x[n] is obtained:

$$AMDF ( k ) = ( \frac{1}{N} ) {\sum }_{n} \vert x [ n ] - x [ n - k ] \vert \qquad [ 9 ]$$

where N is the number of samples in the current analysis window. The AMDF function exhibits a minimum at the period K of the sound signal x[n].

Typically, a window of duration of one minute is sufficient to estimate the breathing rate. Typically, sound signals can be sampled with a sampling frequency of 8 kHz or higher; however, since the breathing period of a person is between 0.5 seconds and 2 seconds at rest or during sleep, the 8 kHz sampling rate is not necessary. Thus, down-sampling the sound data to compute the AMDF function to save computational efficiency may be performed.

Periodicity for regular sound sleep may be observed. Whenever the person exhibits sleep apnea or stops breathing a dip in the AMDF function cannot be observed. At that moment the processor analyzing the data can generate an alarm (e.g., a visual or audible alarm to warn healthcare professionals of a problem). For example, a respiration rate under 12 or over 25 breaths per minute while resting is considered abnormal. If a breathing rate above these limits is detected, an alarm can be generated. As can be seen from FIG. 5 (a) breathing rate can be estimated not only from the sound signal but also from the PIR sensor signal. The low-pass filtered version of the recorded PIR signal is shown in FIG. 5 (b). The fundamental frequency of the PIR sensor signal can be estimated in many ways either from the raw signal shown in FIG. 5(a) or from the low-pass filtered signal shown in FIG. 5(b) by those who are skilled in the art of signal processing. For example, the Fourier transform of the PIR signal can be computed and from the peak of the Fourier transform the fundamental period can be estimated. Time domain methods can be also used to estimate the period, as would be similarly apparent to one of ordinary skill in the art.

For example, to detect respiration rate accurately, the fundamental frequency of the oscillation of PIR signals can be estimated by fitting a periodic curve to the PIR signal. A curve fitting algorithm can be applied to the raw PIR signal and the frequency of the fitted signal can be computed to obtain the respiration rate. Alternatively, the PIR signal can be low-pass or band-pass filtered with a bandwidth corresponding to 10 to 25 periods per minute, for example, which covers 12 to 20 breaths per minute in certain instances. After band-pass filtering the fundamental frequency can be estimated to determine respiration rate.

### Heart-Beat Rate Detection

A normal resting heart-beat rate for adults ranges from 60 to 100 beats a minute. In general, a lower heart rate at rest implies more efficient heart function and better cardiovascular fitness. Heart-beat is also a periodic activity but its period is different than breathing. Therefore, the AMDF function can be used to detect the heart-beat. However, the minima of the AMDF function is searched at different periods than the respiration rate.

Respiration sounds are much stronger than heart-beat rate sound and can interfere with a heart-beat sound. An adaptive noise cancellation algorithm to subtract respiration sounds from the recorded sound signals may be used. Multiple microphones 220, for example, embedded into the blanket may be used. Therefore, the sound data obtained from microphones 220 not facing the subject can be adaptively subtracted from the microphones facing the subject, using e.g., well-known adaptive LMS algorithm. The residual signal may be fed to the AMDF algorithm for heart-beat detection.

### Wheezing Sound Detection

A wheeze is formally called sibilant rhonchi in medicine. It is a continuous, coarse, whistling sound produced during breathing. The American Thoracic Society defines a wheeze sound as an acoustic signal whose dominant frequency is at 400 Hz lasting over 250 ms. Wheezing is caused by obstructions in the respiratory canal and is often a symptom of serious conditions and asthma. Therefore, timely detection of wheezing during sleep may be medically very important.

Detecting a wheeze in a breathing signal can be carried out in various ways. Microphones 220 of the present disclosure can easily pick up the wheeze sounds. The obstruction in the respiratory canal causes a quasi-harmonic behavior in the sound signal. Because of this quasi-harmonic nature, time-frequency techniques have difficulty in yielding efficient and consistent real-time algorithms, the sound data may be divided into a plurality of windows of length 100 ms, for example, because a typical wheeze lasts about 250 ms. Other window durations can be also used. The sound signal is sampled at 8 kHz, but can also be sampled at higher rates such as 16 kHz. In a window of 100 ms there are L=800 samples at 8 kHz sampling rate.

Wheezing may also be detected using Goertzel's algorithm. Goertzel's algorithm is computationally more efficient than Fast Fourier Transform (FFT) when the DFT is computed at a single frequency. Assume L sound samples in a given window of duration 100 ms. First, s[n] can be computed for, n=0, 1, 2, . . . , L, using the formula:

s[n]=x[n]+2 cos(ω_{c})s[n−1]−s[n−2], [10]

where s[−1]=0 and s[−2]=0, and ω_{c} is the normalized angular frequency of 2710.1 corresponding to 400 Hz.

Next, after this recursive operation, compute y[L]:

y[L]=s[L]−e^{-jωc}s[L−1] [11]

Note that: y[L]=X(e^{jωc})e^{jωc}=(Σ^{L}ₙ₌₀x[n]e^{-jωc})e^{jωc}, Thus |y[L]|=|X(e^{jωc})|, X(e^{jωc})=y[L]e^{-jωc}.

Thus, the computational cost of Goertzel's Algorithm is L real multiplications and one complex multiplication. Actually, only the magnitude of the Fourier Transform at ω_{c} is needed. Therefore, it is enough to compute |y[L]| for certain purposes. Goertzel's Algorithm is computationally faster than direct computation of Fourier Transform at ω_{c}X(e^{jωc}) which requires L+1 complex multiplications. It also may be faster than FFT because FFT computes all the DFT coefficients in one shot.

The algorithm of the present disclosure monitors |y[L]| in each window of sound data. Whenever it exceeds a predetermined threshold it means that the subject is wheezing. Goertzel's algorithm at another frequency (e.g. 800 Hz) can be also computed. The two magnitudes may be compared before reaching a final decision. The magnitude at 800 Hz should be much smaller than the magnitude of the Fourier transform at 400 Hz during a wheeze. For example, whenever the Goertzel's algorithm computed at 400 Hz exceeds a predetermined threshold the subject is having a wheeze because this indicates that an abnormal high-frequency activity exists in the recorded sound signal due to wheezing.

Adaptive noise cancellation can be implemented. Let x[n] be the sound signal recorded by the microphone 220 recording the ambient sounds including breathing, snoring and other noises. Index n represents the n-th sound sample obtained at time t=nT where T is the sampling period which can be selected as 1/8000 seconds, for example. Let v[n] be the sound coming from the microphone touching the sleeping person's body. The signal v[n] includes the heart beat sound. It also includes breathing, snoring and other noises. Therefore, one can subtract x[n] from v[n] to obtain the heart-beat sounds, but the amplitude levels may be different and there may be misalignment problems during straightforward subtraction operation. Let b[n] be the estimated heart beat sound at time t=nT. The heartbeat sound can be estimated as follows:

b[n]=v[n]−gₙ(x[n],x[n−1], . . . ,x[n−K]) [12]

where the function gₙ is an adaptive function adjusting the samples and amplitudes.

In adaptive noise cancellation, the function gₙ is a transversal or Finite-extent Impulse Response (FIR) filter, i.e.,

$${g}_{n} ( x [ n ] , x [ n - 1 ] , … , x [ n - K ] ) = {\sum }_{k = 0}^{K} {a}_{n , k} x [ n - k ] \qquad [ 13 ]$$

where the weights a_{n,k} are adaptively determined using the well-known Least-Mean Square (LMS) algorithm by minimizing the mean-square error (MSE)

MSE=E[b[n]²] [14]

using the stochastic gradient algorithm in a recursive manner. The next set of weights

a_{n+1,k}=a_{n,k}−μ∇b[n]²,k=0,1, . . . ,K [15]

where μ is the adaptation parameter, which is usually selected as a small number strictly greater than zero. The stochastic gradient algorithm iteratively finds weights that minimize the MSE=E[b[n]²]. Sound samples coming from secondary microphones x[n], x[n−1], . . . , x[n−K] monitoring the ambient sounds may only contain breathing sounds and ambient noise. Therefore, by minimizing the MSE one will eventually end up with the heart beat sounds in

v[n]−Σₖ₌₀^{K}a_{n,k}x[n−k] [16]

In addition, or as an alternative, to microphones 220, a multisensory approach could include accelerometer(s) 240 (or other vibration sensors) designed to measure vibrations are either based on the piezoelectric effect or electromechanical energy conversion. They are transducers for measuring the dynamic acceleration of the object they are placed. They convert vibrations into electrical signals depending on the intensity of the vibration waves in the axis of the vibration sensor. An accelerometer 240 placed within the blanket 200, or onto the mattress of the subject 300 can continuously monitor him or her during sleep. Whenever the patient moves or unable to lie still high valued accelerometer readings can be recorded.

According to one embodiment, the accelerometer 240 data x(t) of a regular person can be compared to the data of a person with sleeping disturbance. This comparison can produce a number Da(x(t)) indicating the deviation from the normal case. The accelerometer data x(t) is a function of time and it can be a vector covering motion in x, y and z dimensions. Whenever the patient wakes up in the middle of the night and starts wandering, for example, the accelerometer data will exhibit high values just before standing up. Such time instances will be also marked. In such instances, an alarm may be generated and output at/to a personal device of a healthcare professional, for example, to indicate such movement.

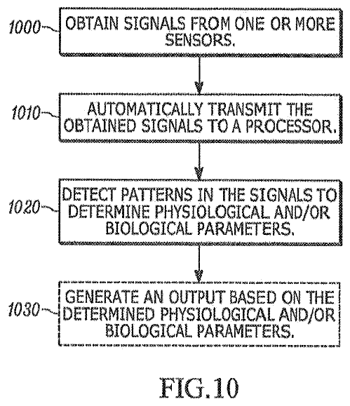

FIG. 10 is a flowchart illustrating an exemplary method of monitoring a subject 300 during a rest or sleep state. At step 1000, signals are obtained from at least one of one or more microphones 220, one or more PIR sensors 100 and one or more accelerometer sensors 240. As discussed herein, the signals captured by the sensors can be automatically transmitted to a processor, at step 1010. The processor may be a remote work station, a handheld device, a personal computer, or may be incorporated in an apparatus communicatively coupled to the one or more sensors. The transmission can be performed using various types of wireless communication, as would be readily apparent to one of ordinary skill in the art.

From step 1010, the process moves to step 1020, where, using the processor, one or more patterns in the obtained signals are detected to determine one or more physiological and/or biological parameters. Here, signals indicating movement and/or sounds of breathing and/or heart beats can be used to determine various parameters including at least one of heart rate, breathing rate, wheezing and/or sleep quality.

Optionally, at step 1030, the processor (or an alternative processor) can generate an output based on the determined parameters. The output can be least one of an audio output, a visual output, an alert message, a report or any combinations thereof, upon determining that values of the determined said physiological and biological parameters are beyond corresponding thresholds, if so desired.

Embodiments described herein provide a nonintrusive methods and systems for monitoring a subject in a sleep state, for example. Tracking sleep using embodiments described herein allows for a determination of whether a subject is sleeping enough and whether a subject's sleep disturbances are a result of a sleep disorder. Understanding the sleep architecture and measuring the sleep quality is the first step in diagnosis and cure of sleep disorders or underlying causes.

Methods described herein may be implemented as software and executed by a general-purpose computer. For example, such a general-purpose computer may include a control unit/controller or central processing unit (“CPU”), coupled with memory, EPROM, and control hardware. The CPU may be a programmable processor configured to control the operation of the computer and its components. For example, CPU may be a microcontroller (“MCU”), a general-purpose hardware processor, a digital signal processor (“DSP”), an application specific integrated circuit (“ASIC”), field programmable gate array (“FPGA”) or other programmable logic device, discrete gate or transistor logic, discrete hardware components, or any combination thereof designed to perform the functions described herein. A general-purpose processor can be a microprocessor, but in the alternative, the processor can be any processor, controller, or microcontroller. A processor can also be implemented as a combination of computing devices, for example, a combination of a DSP and a microprocessor, a plurality of microprocessors, one or more microprocessors in conjunction with a DSP core, or any other such configuration. Such operations, for example, may be stored and/or executed by an onsite or remote memory.

In some embodiments, the methodologies described herein are modules that may be configured to operate as instructed by a general process computer. In the case of a plurality of modules, the modules may be located separately or one or more may be stored and/or executed by the memory unit.

While not specifically shown, the general computer may include additional hardware and software typical of computer systems (e.g., power, cooling, operating system) is desired. In other implementations, different configurations of a computer can be used (e.g., different bus or storage configurations or a multi-processor configuration). Some implementations include one or more computer programs executed by a programmable processor or computer. In general, each computer may include one or more processors, one or more data-storage components (e.g., volatile or non-volatile memory modules and persistent optical and magnetic storage devices, such as hard and floppy disk drives, CD-ROM drives, and magnetic tape drives), one or more input devices (e.g., mice and keyboards), and one or more output devices (e.g., display consoles and printers).

While the invention has been described in terms of several embodiments, those skilled in the art will recognize that the invention is not limited to the embodiments described, can be practiced with modification and alteration within the spirit and scope of the appended claims. The description is thus to be regarded as illustrative instead of limiting.

1. A method of monitoring a subject in a sleep or resting state, comprising:
obtaining signals from at least one of one or more microphones, one or more pyroelectric infrared (PIR) sensors and one or more accelerometer sensors;
automatically transmitting the obtained signals to a processor; and
using the processor, detecting one or more patterns in the obtained signals to determine one or more physiological and/or biological parameters including at least one of heart rate, breathing rate, wheezing, sleep quality and/or sleep architecture.

2. The method of claim 1, wherein at least a subset of the at least one of the one or more microphones, one or more PIR sensors and one or more accelerometers sensors are incorporated as part of a blanket used by the subject.

3. The method of claim 1, wherein the one or more microphones detect ambient sounds as well as sounds from the subject.

4. The method of claim 1, further comprising:
adaptively subtracting, using a noise cancellation algorithm, the obtained sound signals of two or more microphones from each other to extract components corresponding to respiration sounds from the aggregate sound signal; and
determining the breathing rate of the subject using the respiration sounds.

5. The method of claim 1, wherein wheezing is detected using Goertzel's algorithm.

6. (canceled)

7. (canceled)

8. (canceled)

9. (canceled)

10. The method of claim 1, wherein the one or more accelerometer sensors generate an analog time-varying signal according to the motion of the subject's body.

11. (canceled)

12. (canceled)

13. (canceled)

14. The method of claim 1, further comprising generating at least one of an audio output, a visual output, an alert message, a report or any combinations thereof, upon determining that values of the determined physiological and biological parameters are beyond corresponding thresholds.

15. (canceled)

16. A system for monitoring a subject in a sleep or resting state, comprising:
at least one of one or more microphones, one or more pyroelectric infrared (PIR) sensors and one or more accelerometer sensors obtaining signals; and
a wireless transmitter automatically transmitting the obtained signals to a processor, wherein
the processor is configured to detect one or more patterns in the obtained signals to determine one or more physiological and/or biological parameters including at least one of heart rate, breathing rate, wheezing, sleep quality and/or sleep architecture.

17. The system of claim 16, wherein at least a subset of the at least one of the one or more microphones, one or more PIR sensors and one or more accelerometers sensors are incorporated as part of a blanket used by the subject.

18. The system of claim 16, wherein the one or more microphones detect ambient sounds as well as sounds from the subject.

19. (canceled)

20. (canceled)

21. (canceled)

22. (canceled)

23. (canceled)

24. (canceled)

25. The system of claim 16, wherein the one or more accelerometer sensors generate an analog time-varying signal according to the motion of the subject's body.

26. (canceled)

27. (canceled)

28. (canceled)

29. The system of claim 16, wherein the processor is further configured to generate at least one of an audio output, a visual output, an alert message, a report or any combinations thereof, upon determining that values of the determined physiological and biological parameters are beyond corresponding thresholds.

30. (canceled)

31. A system for monitoring a subject in a sleep or resting state, comprising:
a blanket used by the subject and including at least one of one or more microphones and one or more accelerometer sensors;
one or more pyroelectric infrared (PR) sensors; and
a wireless transmitter communicatively coupled to the blanket and the one or more PIR sensors automatically transmitting obtained signals from the at least one of the one or more microphones, the one or more accelerometer sensors and the one or more PR sensors to a processor, wherein
the processor is configured to detect one or more patterns in the obtained signals to determine one or more physiological and/or biological parameters including at least one of heart rate, breathing rate, wheezing, sleep quality and/or sleep architecture.

32. (canceled)

33. The system of claim 31, wherein the processor is further configured to:
adaptively subtract, using a noise cancellation algorithm, the obtained sound signals of two or more microphones from each other to extract components corresponding to respiration sounds from the aggregate sound signal; and
determine the breathing rate of the subject using the respiration sounds.

34. (canceled)

35. (canceled)

36. (canceled)

37. (canceled)

38. (canceled)

39. (canceled)

40. (canceled)

41. (canceled)

42. (canceled)

43. The system of claim 31, wherein the processor is further configured to generate at least one of an audio output, a visual output, an alert message, a report or any combinations thereof, upon determining that values of the determined physiological and biological parameters are beyond corresponding thresholds.

44. (canceled)

45. A blanket for monitoring a subject in a sleep or resting state, comprising:
at least one of one or more microphones, and one or more accelerometer sensors obtaining signals when the blanket is used by the subject; and
a wireless transmitter automatically transmitting the obtained signals to a processor, wherein
the processor is configured to detect one or more patterns in the obtained signals to determine one or more physiological and/or biological parameters including at least one of heart rate, breathing rate, wheezing, sleep quality and/or sleep architecture.

46. The blanket of claim 45, wherein the one or more microphones detect ambient sounds as well as sounds from the subject.

47. (canceled)

48. (canceled)

49. (canceled)

50. (canceled)

51. (canceled)

52. (canceled)

53. The blanket of claim 45, wherein the processor is further configured to generate at least one of an audio output, a visual output, an alert message, a report or any combinations thereof, upon determining that values of the determined physiological and biological parameters are beyond corresponding thresholds.

54. An apparatus for monitoring a subject in a sleep or resting state, comprising:
one or more pyroelectric infrared (PR) sensors; and
a wireless transmitter communicatively coupled to the One or more PIR sensors automatically transmitting obtained signals from the one or more PIR sensors to a processor, wherein
the processor is configured to detect one or more patterns in the obtained signals to determine one or more physiological and/or biological parameters including at least one of heart rate, breathing rate, wheezing, sleep quality and/or sleep architecture.

55. (canceled)

56. (canceled)

57. (canceled)

58. (canceled)

59. (canceled)

60. The apparatus of claim 54, wherein the processor is further configured to generate at least one of an audio output, a visual output, an alert message, a report or any combinations thereof, upon determining that values of the determined physiological and biological parameters are beyond corresponding thresholds.

61. (canceled)

62. (canceled)
